(19) United States
(12) Patent Application Publication
(10) Pub. No.: US 2004/0072140 A1
(43) Pub. Date: Apr. 15, 2004

(54) Dehydrated antigen presenting cells usable for vaccination

(76) Inventors: Jacques Bartholeyns, Paris (FR); Didier Prigent, Bures sur Yvette (FR)

Correspondence Address: YOUNG & THOMPSON, 745 SOUTH 23RD STREET 2ND FLOOR, ARLINGTON, VA, 22202

(21) Appl. No.: 10/433,960

(22) PCT Filed: Nov. 19, 2001
(86) PCT No.: PCT/EP01/13354

(30) Foreign Application Priority Data
Dec. 7, 2000 (EP) 00403429.4

(51) Int. Cl. A01N 1/02; C12N 5/08

(52) U.S. Cl. 435/2; 435/372

(57) ABSTRACT

The invention relates to a dehydrated antigen presenting cells obtained from initial fresh antigen presenting cells and being liable to generate an immune response against the same antigen(s) as the one(s) against which the initial antigen presenting cells are directed.

The invention relates to new dehydrated antigen presenting cells, a process for their preparation and their use for the preparation of cellular vaccines.

For more than 50 years, freeze-drying has been the most effective method of stabilizing and preserving a wide range of products such as food, chemical or pharmaceutical products.

Antigen presenting cells (APCs) are potent for the priming of an immune response against a specific exogenous antigen by the stimulation of lymphocytes. Their use is a promising tool for the development of cellular vaccines. Among professional APCs, dendritic cells and macrophages ingest, process and present antigens to T cells in association with MHC class I and MHC class II cell surface glycoproteins. Activation of T cells is also stimulated by costimulatory molecules, present on the cells surface. With the growing interest in cellular immunotherapy and cellular vaccines, there is a need for convenient availability of cells able to stimulate the immune system, and particularly to stimulate an immune response against specific antigens.

During a cell therapy process, cells are taken from a patient, for example by blood apheresis, then differentiated into antigen presenting cells and cultured ex vivo under specific conditions (for example according to WO 94/26875, WO 96/22781 or WO 97/44441) and re-injected to the patient. This process can be done extemporaneously, with the re-injection of fresh cells to the patient. However, the use of fresh cells requires immediate cell preparation each time it is necessary, with only temporary conservation at 4° C. The cells may also be frozen and thawed just before use. Freezing cells implies to control the freezing procedure. Furthermore, the storage and transportation of frozen cells needs adequate material and conditions, and consecutive expenses are significant.

U.S. Pat. No. 5,059,518 describes lyophilised mammalian cells able to be reconstituted to exhibit structural and cell surface antigens. Such cells are prepared in order to be used as analytical control in cytofluorimetry analysis. A sugar trehalose solution is used as a preservative or protective agent on the exterior surface of the cells but, according to the applicants, trehalose may not be a useful additive in all situations and its toxicology is to be noted. In particular, these products cannot be administered to patients.

Cellular vaccines represent a new emerging field, using mainly antigen presenting cells. Freeze drying of cells presenting antigens has not been envisaged for these sensitive eucaryotic cells which are believed until now to lose all their stimulatory properties during drying.

The aim of the present invention is to provide ready to use and immunogenic antigen presenting cells usable for immunotherapy and for vaccinology. This aim is achieved by new dehydrated antigen presenting cells obtained from initial fresh antigen presenting cells and being liable to generate an immune response against the same antigen(s) as the one(s) against which the initial antigen presenting cells are directed.

The cells according to the invention are usable for the administration to a patient, whereas being adapted to long term storage at ambient temperatures in a cost efficient manner and keeping intact fresh cells morphological characters important for efficient vaccination.

The term “dehydrated cells” means that the cells have lost more than about 40% of their constitutive water subsequently to a drying process. The proportion of residual water being comprised from about 50 to 1% of the initial cellular water.

The expression “able to generate an immune response” means that the antigen presenting cells according to the invention are able to stimulate or to inhibit an immune response. A stimulated immune response might be characterized, in vivo, by a clinical immune response against a given pathogen or a tumour, leading to its decrease or its elimination. In vitro, it may be measured, for dendritic cells, in an immunostimulation assay of T lymphocytes specific for a given antigen. Treated macrophages can be assessed for their adherence to particular antigens or to tissues expressing defined antigens, and for their release of cytokines and chemokines. Some macrophages may be targeted to specific antigens, for example by antibodies binding either to the antigen and to the Fc receptor on macrophages membranes; the functionality of these cells treated according to the invention may be measured as their target recognition capacity and by an analysis of their cytokine and chemokine release. An inhibited immune response might be observed clinically, in the case of an auto-immune disease, by the decrease or disappearance of the symptoms. In vitro, antigen presenting cells able to inhibit an immune response are characterized by their decreased secretion of stimulatory cytokines (IL-1, IL-12, IFN-γ) and their increased secretion of certain inhibiting cytokines (IL 10, TGF-β).

The expression “usable for the administration to a patient” means that the cells and additives are of clinical grade.

According to an embodiment of the invention, the dehydrated antigen presenting cells present on their surface MHC class I, MHC class II and co-stimulatory molecules. According to one particular embodiment of the invention, dehydrated antigen presenting cells present on their surface MHC class I and MHC class II molecules, with CD 16, CD64 and CD45 molecules. According to another particular embodiment of the invention, dehydrated antigen presenting cells present on their surface MHC class I and MHC class II molecules, with CD40, CD80 and CD86 co-stimulatory molecules.

In a preferred embodiment of the invention, dehydrated antigen presenting cells present previously interiorised or adsorbed antigenic peptides on their surface, in association with MHC class I and/or MHC class II molecules.

According to an other embodiment of the invention, the dehydrated antigen presenting cells are able to induce proliferation of T cells in vitro, as measured in Mixed Lymphocyte Reactions (MLR).

According to an other embodiment of the invention, dehydrated antigen presenting cells are able to induce in vivo proliferation of antigen-specific T cells.

In an embodiment of the invention, dehydrated antigen presenting cells are blood cells, cells derived from blood cells, or blood stem cells or somatic cells.

In a particular embodiment of the invention, dehydrated antigen presenting cells are monocyte derived cells. In a more particular embodiment of the invention, dehydrated antigen presenting cells are monocyte derived macrophages. The monocyte derived macrophages may be directed towards an antigen by an antibody binding either to an Fc receptor located on the surface of the macrophage and to the antigen. In an other particular embodiment of the invention, dehydrated antigen presenting cells are monocyte derived dendritic cells.

According to an other embodiment of the invention, dehydrated antigen presenting cells are dehydrated macrophages which have preserved capacity of fresh macrophages to bind to specific cells, tissues, or antigens, in vitro or in vivo, and to deliver to this site an agent which may have a therapeutic effect.

A general method of preparation of dehydrated antigen presenting cells obtained from initial fresh antigen presenting cells and being liable to generate an immune response against the same antigen(s) as the one(s) against which the initial antigen presenting cells are directed, which comprises the following step:

a Sublimation of ice contained in a frozen cellular preparation of fresh antigen presenting cells under low pressure conditions.

More particularly, a general method of preparation of dehydrated antigen presenting cells according to the invention comprises the following steps:

Preparation of the antigen presenting cells

Freezing of the cells

Sublimation of the ice contained in the frozen cellular preparation under low pressure conditions.

In a particular method of preparation of dehydrated antigen presenting cells according to the invention, for example, the cells are frozen under temperature comprised from about −20 to about −180° C. and at atmospheric pressure, in a process such as lyophilisation. The sublimation of ice may be achieved in an enclosure in which the inside pressure is less than about 6 mbar (600 Pa).

An other method of preparation of dehydrated antigen presenting cells according to the invention comprises the following steps:

freezing of the antigen presenting cells preparation under the conjugated action of low temperature and low pressure conditions

sublimation of the ice contained in the frozen preparation under low pressure conditions

The product must be maintained at a temperature of about −20° C. during dehydration. Whereas the temperature is usually lowered in the enclosure by the circulation of a cold fluid, a person skilled in the art may also decrease the temperature within the product by evaporating about 15 to about 20% of the water contained within the product, under vacuum. As an example, cells may be frozen at temperature decrease from room temperature (about +20° C.) to about −20° C., concomitantly with pressure diminution from atmospheric pressure to about 1 mBar. The combination of these conditions leads to the freezing of the cells and to their partial dehydration (from about −15% to −20% of water). After being frozen, the internal pressure of the enclosure is put at a value of less than about 6 mbar, in order to complete cell dehydration.

In a particular embodiment of the invention, the sublimation of the ice is performed under low pressure conditions, in an enclosure linked to a water trap made of a crystallized clay, greedy of water vapour and having a pore diameter such as to trap water molecules only. In a preferred embodiment of the invention, the crystal has a pore diameter of about 1 to about 5 Angstrom, the crystal pore diameter comprised from about 1 to about 5 Angstrom implies that only water molecules (about 3 Angstrom diameter) are trapped. Molecules having a diameter larger than about 5 Angstrom are preserved within the product. In a preferred embodiment of the invention, the crystal may be an organic clay, such as argil or terracotta, or a crystallized clay with adequate pore diameter, such as talc, mica, shist, or zeolite.

The antigen presenting cells according to the invention are dehydrated under sterile and controlled atmosphere. In a preferred embodiment of the invention, the dehydration of the cells is performed on cells enclosed in an porous sterile container, which may be a bag and particularly a culture bag. In a preferred embodiment of the invention, the cells are subsequently cultured, possibly treated or antigen loaded, and dehydrated in the same bag.

Monocyte derived cells can be prepared according to patent applications WO 94/26875, WO 96/22781 or WO 97/44441 for example.

Advantageously, dehydrated antigen presenting cells according to the invention have been loaded with at least one antigen prior being dehydrated. In an embodiment of the invention, antigen presenting cells have been antigen loaded by phagocytosis, pinocytosis, affinity, fusion, nucleic acid (DNA, RNA) transfer or receptor mediated uptake, according to methods known by a man skilled in the art.

In a particular embodiment of the invention, dehydrated antigen presenting cells result from the fusion of antigen presenting cells and tumoral cells. These cells might haven been fused with methods based on the use of compounds such as polyethylene glycol, or by electrofusion.

The present invention also concerns a method of preparation of rehydrated antigen presenting cells comprising the addition of a resuspension solution to the cells, the cell preparation and the resuspension solution being at the same temperature, for example at room temperature (about 20° C.). The resuspension solution is progressively and gently added to the cell preparation present as a friable powder. The rehydration of the cells lead to a liquid mixture able to be administered to patients, for example by injection. The present invention also concerns rehydrated antigen presenting cells prepared according this method of rehydration. The invention further concerns cellular vaccine compositions or immunotherapeutic drugs containing, as active substance, rehydrated antigen presenting cells prepared according to the method of rehydration.

In a particular embodiment of the invention, a method of preparation of antigen presenting cells to be dehydrated comprises the following steps:

Isolation of leucocytes from peripheral blood by apheresis, from healthy donors or from patients,

Culture of the mononuclear cells by placing them in an appropriate culture medium containing chemical ligands of mononuclear cells, for a time sufficient to obtain differentiated monocyte-derived antigen presenting cells (MD-APC), according to protocols described in WO 94/26875, WO 96/22781 or WO 97/44441.

Recovering the monocyte derived antigen presenting cells.

The present invention also relates to a method of preparation of antigen presenting cells to be dehydrated, the culture medium being completed with soluble or particulate antigens, including target cells or cell debris, or specific peptides against which an immune response is desired. In an other particular embodiment of the invention, the culture medium is added with genetic material coding for a peptide or a protein against which an immune response is desired, linked to a vector able to allow the transfection of the MD-APCs.

The present invention relates to dehydrated antigen presenting cells liable to be obtained according to the previously described processes.

Dehydrated antigen presenting cells liable to be obtained by these processes of preparation are conditioned in a medium containing from about 0 to about 100% of autologous serum, and preferably from about 10 to about 85% of serum. The presence of autologous serum stabilizes proteins, lipoproteins and membranes structures.

In a preferred embodiment of the invention, dehydrated antigen presenting cells are conditioned in a medium containing glucose at concentrations varying from about 0 to about 10% of the volume, to preserve glycolipids and glycoproteins during the process.

In a preferred embodiment of the invention, dehydrated antigen presenting cells are conditioned in a medium containing human serum albumin at concentrations varying from about 0 to about 20% of the volume, to stabilize glycoproteins.

In another preferred embodiment of the invention, dehydrated antigen presenting cells are conditioned in a medium containing dimethylsulfoxide (DMSO), used as a cryopreservative compound, at concentrations varying from about 0 to about 10% of the volume.

In a preferred embodiment of the invention, dehydrated antigen presenting cells liable to be obtained by these processes of preparation are conditioned in a medium free from cryopreservation components that should preferably have been eliminated before administration of the cells to patients. Therefore, in this case, the dehydrated cells have only to be reconstituted with water before administration, without any cells washing. This particularity leads to a quicker and more simple preparation of the dehydrated cells to be administered, decreasing the manipulations and the possibility of contamination of the cell preparation.

The present invention also relates to pharmaceutical compositions containing, as active substance, dehydrated or rehydrated antigen presenting cells obtained from initial fresh antigen presenting cells and being liable to generate an immune response against the same antigen(s) as the one(s) against which the initial antigen presenting cells are directed.

The present invention also relates to cellular vaccine compositions or immunotherapeutic drugs containing, as active substance, dehydrated or rehydrated antigen presenting cells obtained from initial fresh antigen presenting cells and being liable to generate an immune response against the same antigen(s) as the one(s) against which the initial antigen presenting cells are directed.

Pharmaceutical compositions, cellular vaccine compositions or immunotherapeutic drugs might be administered to patients under various galenic forms comprising the intradermal, subcutaneous, sublingual, intraveinous, intralymphatic, intranodal or intramuscular administration containing dehydrated or rehydrated antigen presenting cells prepared according to the methods described. The number of cells in a single dose of dehydrated or rehydrated cells according to the invention being comprised from about 10⁶ to about 10⁹ cells for a patient, and preferably from about 10⁷ to about 10⁸ cells for a patient.

### DESCRIPTION OF THE FIGURES

FIGS. 1A, 1B, 1C: Phenotypic FACS analysis of dendritic cells after dehydration and re-hydration (hollow curves) as compared to fresh dendritic cells (black curves). The dotted line corresponds to a negative control.

FIG. 1A represents the phenotypic analysis of HLA DR (CMH Class II molecules), FIG. 1B represents the phenotypic analysis of costimulation molecule CD80, FIG. 1C represents the phenotypic analysis of costimulation molecule CD40.

The X axis represents the intensity of fluorescence detected on the surface of the cells, the Y axis represent the number of cells in each population. PE and FITC are the fluorescent markers used for the detection of the surface molecules.

Treated and untreated DCs were harvested, washed in PBS and resuspended in PBS supplemented with autologous serum. 100 μl of cell suspension were incubated on ice for 30 min with FITC-conjugated mAb anti-HLADR, PE-conjugated mAb anti-CD80, or PE-conjugated mAb anti-CD40, or with correspondent PE- and FITC-conjugated mouse isotype controls (Immunotech, Marseille, France). Cells were then washed again and resuspended in PBS containing 3 nM of the nucleic acid stain TO-PRO-3 (Molecular Probes, Eugene, Oreg.) to exclude death cells from analysis.

FIGS. 2A, 2B, 2C, 2D: Phenotypic FACS analysis of macrophages after dehydration and re-hydration (hollow curves) as compared to fresh macrophages (black curves). The dotted line corresponds to a negative control.

FIG. 2A represents the phenotypic analysis of HLA DR (CMH Class II molecules), FIGS. 2B and 2C represent the phenotypic analysis of surface receptors CD16 and CD64, FIG. 2D represents the phenotypic analysis of surface molecule CD45.

The X axis represents the intensity of fluorescence detected on the surface of the cells, the Y axis represent the number of cells in each population. PE and FITC are the fluorescent markers used for the detection of the surface molecules.

Treated and untreated mature DC were harvested, washed in PBS and resuspended in PBS at 5.10⁶ cells/ml. 100 gl of cell suspension were incubated on ice for 30 min with PE-conjugated mAb anti-HLADR, PE-conjugated mAb anti-CD16, PE-conjugated mAb anti-CD64, or FITC-conjugated mAb anti-CD45, or with correspondent PE- and FITC-conjugated mouse isotype controls (Immunotech, Marseille, France). Cells were then washed again and resuspended in PBS containing 3 nM of the nucleic acid stain TO-PRO-3 (Molecular Probes, Eugene, Oreg.) to exclude death cells from analysis. Flow cytometry analysis was performed in a FACSCalibur with a CellQuest software.

### EXAMPLES

### Example 1

Preparation of Dehydrated Dendritic Cells

Cells

Antigen presenting cells are elutriated human immature dendritic cells (DCs), prepared by culture of peripheral blood monocytes, according to the patent application WO 97/44441 and to Boyer et al. (“Generation of phagocytic MAK and MAC-DC for therapeutic use: Characterization and in vitro functional properties” Exp. Hematol., 1999, 27, 751-761).

5.10⁷ dendritic cells are suspended in 10 ml PBS with 10% albumin and 10% DMSO, in a 50 ml tube (Falcon). Cells are frozen at −80° C.

Drying of the Cells

The frozen cells sample is placed in an enclosure in which the internal pressure is less than 6 mbar, and the temperature maintained inferior to the dehydration medium freezing point. After 24 hours in these conditions, the enclosure is opened and the samples are taken. The dehydrated product appears as a friable powder.

### Example 2

Rehydration and Analysis of Dehydrated Dendritic Cells

Rehydration of the Cells

Dehydrated dendritic cells prepared as in example 1 are weighted, ⅕ of the cells (10⁷ cells) are solubilized and resuspended in a washing solution (phosphate buffer with glucose, BRAUN), then centrifuged at 1300 rpm, for 7 min, at 4° C. The cells pellet is gently suspended in 2 ml of sterile water (5.10⁶ cells/ml)

Viability, Observation and Adherence Properties

Cells are diluted ½ and counted on Malassez plates. Viability of the cells is assessed by Trypan blue exclusion. The cells morphology is observed after a coloration.

The adherence of living cells is observed on a glass microscope slide (Lab-Tek® II). Cells are suspended in IDM medium supplemented with 2% AB+ serum, (2.10⁵ cells/ml), 1 ml of cell suspension is placed in each of the two wells. The medium is eliminated and cells are washed three times with sterile PBS. Cells are coloured with a May-Grunwal-Giemsa reaction and are observed.

Results

After being frozen and dried, 1 to 10% of rehydrated dendritic cells are viable. All the viable cells adhere on the lame.

Microscopic observation of the cells shows that the cell membrane integrity is preserved.

FACS analysis of the size and granulometry of the cells show that the dehydrated and rehydrated dendritic cells is composed of two populations, one of them conserving the characteristics of the dendritic cells before dehydration treatment.

Phenotypic Analysis

Flow cytometry analysis: DCs are suspended in PBS supplemented with autologous serum 1%, at 4.10⁶ cells/ml. 100 μl of cell suspension (4.10⁵ cells in each tube) were incubated on ice in obscurity for 30 min with fluorochrome conjugated monoclonal antibody: 10 μl of FITC-conjugated anti-HLA DR, 10 μl of PE-conjugated mAb anti-CD80 or 10 μl of PE-conjugated nab anti-CD40 (Immunotech, Marseille, France). Cells were then washed again in non-sterile PBS, centrifuged at 1400 rpm for 5 min at 20° C. and resuspended in PBS.

Flow cytometry analysis was performed in a Becton Dickinson cytometer with a CellQuest software.

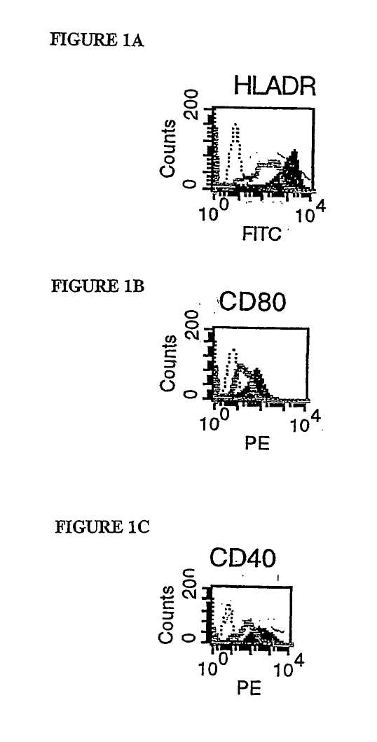

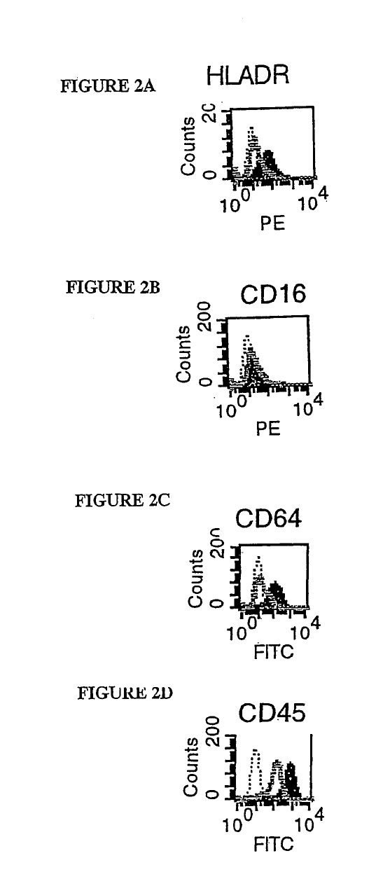

The X axis represents the intensity of fluorescence detected on the surface of the cells, the Y axis represent the number of cells in each population. The white empty signal correspond to the negative control, the full signal corresponds to control DCs, which are DCs frozen and thawed. Results are presented in FIGS. 1A, 1B, and 1C. The figures show a partial conservation of the expression of HLA-DR, CD80, CD86 and CD40 molecules by the dehydrated dendritic cells.

### Example 3

Preparation of Dehydrated Macrophages

Cells

Antigen presenting cells are elutriated non activated human macrophages prepared according to U.S. Pat. No. 5,662,899 and to Boyer et al. (“Generation of phagocytic MAK and MAC-DC for therapeutic use: Characterization and in vitro functional properties” Exp. Hematol., 1999, 27, 751-761).

5.10⁷ cells are suspended in 10 ml PBS with 10% albumin and 10% DMSO, in a 50 ml tube (Falcon). Cells were frozen at −80° C.

Drying

The frozen cells;sample is placed in an enclosure in which the internal pressure is less than 6 mbar, and the temperature maintained inferior to the dehydration medium freezing point. After 24 hours in these conditions, the enclosure is opened and the samples are taken. The dehydrated product appears as a friable powder.

### Example 4

Rehydration and Analysis of Dehydrated Macrophages

Rehydration

Dehydrated macrophages, prepared in example 3, are treated as detailed in example 2.

After freezing and drying, about 6% of rehydrated macrophages are viable. All the viable cells adhere on the lame.

Microscopic observation of the cells shows that the cell membrane integrity is preserved. FACS analysis of the size and granulometry of the cells show that the dehydrated and rehydrated macrophages is composed of several populations, one of them conserving the characteristics of the macrophages before dehydration treatment.

Phenotypic analysis is assessed as described in example 2.

Macrophages are suspended in PBS supplemented with autologous serum 1%, at 4.10⁶ cells/ml. 100 μl of cell suspension (4.10⁵ cells in each tube) were incubated on ice in obscurity for 30 min with fluorochrome conjugated monoclonal antibody: 10 μl of PE-conjugated or FITC-conjugated mAb anti-HLA DR, 10 μl of PE-conjugated mAb anti-CD16, 10 μl of FITC-conjugated mAb anti-CD64, 10 μl of FITC-conjugated mAb anti-CD45 (Immunotech, Marseille, France). Then cells are treated as described in example 2. Results are shown in FIGS. 2A, 2B, 2C and 2D. The figures show a partial conservation of the expression of HLA-DR, CD16, CD45 and CD64 molecules by the dehydrated macrophages.

### Example 5

Preparation of Antigen-Loaded Dessicated Antigen Presenting Cells and In Vitro Stimulation of T Lmphocyte Proliferation by Dessicated and Rehydrated Antigen Presenting Cells

Antigen presenting cells are elutriated human DCs, prepared according to WO 97/44441 and Boyer et al. (“Generation of phagocytic MAK and MAC-DC for therapeutic use: Characterization and in vitro functional properties” Exp. Hematol., 1999, 27, 751-761).

Dendritic Cells Human immature dendritic cells (DCs) are elutriated after 7 days of differentiation in AIMV medium supplemented with 500 U/ml GM-CSF and 50 ng/ml IL13 (complete AIMV medium). Dendritic cells are incubated in AIMV medium for 4 hours in the presence of 0.1 to 1 g/ml of the peptidic antigens to be loaded, or for 16 hours in the presence of 2.10⁶ tumour cell lysates/ml. Culture was done in 24 wells plates with 2.10⁶ DCs/ml.

The cells are washed or further matured as described below and then resuspended at 10⁷ cells/ml in PBS medium containing 85% of autologous serum, 5% glucose and 10% DMSO before freezing and dehydration.

Maturation Conditions

DCs are incubated in complete AIMV medium for 60 hours in the presence of different concentrations of the clinical grade maturation reagents. IFNγ (Imukin) 1000 U/ml, bacterial membrane extracts (Ribomunyl®, 1 to 10 μg/ml), or 3 μg/ml anti-CD40+ 100 μg/ml poly(I:C).

Phenotype Analysis

The following markers were used to follow DCs maturation by FACS: CD14, HLA ABC, CD83, CD86.

Culture Recovery and Cell Viability

Cell recovery after culture was estimated by counting living cells on Malassez slide. Cell viability was measured by FACS using TOPRO-3.

Allogeneic MLR

Variable numbers of DCs were incubated during 5 days with a fixed number of allogeneic T lymphocytes. Cell proliferation during the last 18 hours of culture was quantified by (³H) thym thymidine uptake of cells incubated with 1 μCi of (methyl-3H) thymidine.

Cytokine Detection

Culture supernatants were assayed for IL-12 p70 cytokine detection by ELISA.

Specific Immune Stimulation In Vitro

We test the capacity of rehydrated antigen presenting cells to stimulate autologous T cells specific for a given antigen. These specific stimulations can be generated ex vivo in response to whole influenza virus (Mutagrip from Aventis-Pasteur), to hepatitis B surface antigen (HbS antigen provided by SmithKline Beecham), to the immunodominant peptide of the influenza Matrix antigen, to the melanoma specific tumour antigen MelanA/MART1 and to the prostate specific antigen PSA interiorised by the antigen presenting cells before dehydration.

Results

Dendritic cells matured in culture in the presence of cytokines and of tumour antigens are either kept at 4° C. or dehydrated at −20° C. under low pressure, overnight through organic filters. They are then rehydrated with isotonic solution and used to stimulate immune proliferation of allogeneic T cells and to measure specific immunostimulating potential, as described.

Results show that rehydrated antigen presenting cells keep their potency to stimulate allogeneic T lymphocytes proliferation at 1/10 ratio (10⁴ DC for 10⁵ T cells), preserve a phenotype of mature DCs (CD14−, HLA ABC++, CD83+, CD86++) and release high amount of IL-12 p70.

Using a cocktail of three immunodominant peptides, MelanA/MART1 specific CD8 T cells are obtained and further purified using cognate MHC/peptide tetramers and the resulting population is shown to be CD8+ and able to kill peptide-loaded, autologous EBV-B cells as well as a MelanA/MART1 positive melanoma cell line sharing the HLA-A2 MHC class I molecule. Furthermore, activated T cells secrete IFN-γ as determined by ELISPOT and intracellular cytokine measurement. The antigen presenting cells prepared according to our technology have preserved a vaccinal potency in in vitro models.

1. Dehydrated antigen presenting cells obtained from initial fresh antigen presenting cells and being liable to generate an immune response against the same antigen(s) as the one(s) against which the initial antigen presenting cells are directed.

2. Dehydrated antigen presenting cells according to claim 1 characterized in that they present on their surface MHC class I and MHC class II molecules, with CD16, CD64 and CD45 molecules.

3. Dehydrated antigen presenting cells according to claim 1 characterized in that they present on their surface MHC class I and MHC class II molecules, with CD40, CD80 and CD86 co-stimulatory molecules.

4. Dehydrated antigen presenting cells according to any one of claims 1 or 3 characterized in that cells have been loaded with at least one antigen prior being dehydrated.

5. Dehydrated antigen presenting cells according to any one of claims 1 to 4 characterized in that they present previously interiorised or adsorbed antigenic peptides on their surface, in association with MHC class I and/or MHC class II molecules.

6. Dehydrated antigen presenting cells according to any one of claims 1 to 5 characterized that they are blood cells, cells derived from blood cells, or blood stem cells or somatic cells.

7. Dehydrated antigen presenting cells according to claim 6 chosen within the group consisting of monocyte derived cells, macrophages and dendritic cells.

8. Dehydrated antigen presenting cells according to any one of claims 1 to 7 characterized in that cells result from the fusion of antigen presenting cells and tumoral cells.

9. Dehydrated antigen presenting cells according to any one of claims 1 or 2 characterized in that they are dehydrated macrophages which have preserved capacity of fresh macrophages to bind to specific cells, tissues or antigens, in vitro or in vivo, and to deliver to this site an agent which may have a therapeutic effect.

10. Method of preparation of dehydrated antigen presenting cells obtained from initial fresh antigen presenting cells and being liable to generate an immune response against the same antigen(s) as the one(s) against which the initial antigen presenting cells are directed comprising the following step:
sublimation of ice contained in a frozen cellular preparation of fresh antigen presenting cells under low pressure conditions.

11. Method of preparation of dehydrated antigen presenting cells according to claim 10, in which the freezing of the cells is achieved under the conjugated action of low temperature and low pressure conditions.

12. Dehydrated antigen presenting cells liable to be obtained according to the process of any one of claims 10 or 11.

13. Rehydrated antigen presenting cells obtained from the rehydration of dehydrated antigen presenting cells according to any one of claims 1 to 9.

14. Method of preparation of rehydrated antigen presenting cells according to claim 13 comprising the addition of a resuspension solution to dehydrated cells according to any claim 1 to 9, the dehydrated antigen presenting cells and the resuspension solution being at about the same temperature.

15. Pharmaceutical compositions containing as active substance dehydrated antigen presenting cells, according to claim 1 to 9 or rehydrated antigen presenting cells according to claim 13.

16. Cellular vaccine compositions containing as active substance dehydrated antigen presenting cells, according to claim 1 to 9, or rehydrated antigen presenting cells according to claim 13.
